(19) United States
(12) Patent Application Publication
(10) Pub. No.: US 2025/0189547 A1
(43) Pub. Date: Jun. 12, 2025

(54) SURFACE-ENHANCED RAMAN NANOPARTICLE AND DETECTION METHOD FOR FENTANYLS

(71) Applicant: Shenzhen Customs Industrial Products Testing Technology Center, Shenzhen (CN)

(72) Inventors: Tangtang XIE, Shenzhen (CN); Wei PENG, Shenzhen (CN); Junfeng LIN, Shenzhen (CN); Yanhua LI, Shenzhen (CN); Jianfeng LI, Shenzhen (CN)

(73) Assignee: Shenzhen Customs Industrial Products Testing Technology Center, Shenzhen (CN)

(21) Appl. No.: 18/591,005

(22) Filed: Feb. 29, 2024

(30) Foreign Application Priority Data
Dec. 6, 2023 (CN) 2023116736279

(51) Int. Cl. G01N 33/94; B82Y 15/00; G01N 1/28; G01N 21/65

(52) U.S. Cl. CPC G01N 33/9486; G01N 1/28; G01N 21/658; B82Y 15/00

(57) ABSTRACT

The present invention focuses on a surface-enhanced Raman nanoparticle and its application in detecting fentanyls, within the realm of drug safety assessment. The surface-enhanced Raman nanoparticle comprises a noble metal, with gold nanocakes being the preferred choice. The detection method for fentanyls employs the surface-enhanced Raman nanoparticle described in the present invention to conduct Raman detection. This nanoparticle and the associated detection method enable the rapid and non-destructive identification of trace amounts of fentanyls in the target sample, presenting significant potential for application in public safety detection.

### CROSS-REFERENCES

This application claims the priority benefit of Chinese Patent Application Serial Number 2023116736279, filed on Dec. 6, 2023, the full disclosure of which is incorporated herein by reference.

### TECHNICAL FIELD

The present invention relates to the field of drug safety detection, and specifically focuses on a surface-enhanced Raman nanoparticle sol and a detection method for fentanyls.

### BACKGROUND OF THE INVENTION

Fentanyl is a potent synthetic opioid and a highly effective synthetic anesthetic, classified within the opioid drug category. In recent years, illegal production and distribution of fentanyl have escalated, resulting in its widespread abuse and emergence as a significant global public health issue. Numerous countries have intensified regulatory efforts and undertaken measures to combat the illicit production and distribution of fentanyl.

The customs system, serving as the primary line of defense at the national border, must conduct comprehensive and precise inspections of import and export goods and packages to prevent the illicit movement of articles across the border. Fentanyl, a highly perilous drug, poses a significant threat to society and public safety if not promptly and efficiently identified and intercepted. Crucially, the non-destructive detection of fentanyl in mail and packages is imperative. Given the substantial volume of mail and packages, manual inspection is both time-consuming and labor-intensive, potentially resulting in damage to the packages. Non-destructive detection technology not only enhances detection speed and efficiency but also minimizes damage to mail and packages, thereby reducing postal operational costs and augmenting customs detection capabilities.

Nevertheless, the chemical structure of fentanyl is inherently intricate, with numerous derivatives. Conventional methods, such as mass spectrometry, demand specialized instruments and skilled technicians, resulting in high costs and prolonged analysis times. Drug detection belts and canine olfaction, while widely employed, are susceptible to issues like disguise and misjudgment. Additionally, manually simulating characteristics of suspicious articles may lead to misjudgments. Furthermore, X-ray imaging technology exhibits limited efficacy in detecting novel fentanyl derivative drugs.

Therefore, it is crucial to develop a technically simple and convenient means to achieve rapid and non-destructive detection of fentanyl and its derivative drugs in mail and packages.

### SUMMARY OF THE INVENTION

The present invention introduces a novel surface-enhanced Raman nanoparticle, a corresponding sol, and a fentanyl detection method. The objective is to address the challenges prevalent in existing fentanyl detection methods, such as complex procedures, low efficiency, high costs, packaging damage, reduced accuracy, poor sensitivity, and incomplete detection.

In a first aspect, the present invention introduces a surface-enhanced Raman nanoparticle preferably featuring a gold nanocake structure. This nanoparticle exhibits rapid, accurate, and highly sensitive detection of fentanyls at a low cost, in a non-destructive manner. Furthermore, it is capable of detecting multiple types of fentanyls, demonstrating exceptional technical efficacy.

In a second aspect, the present invention offers a surface-enhanced Raman nanoparticle sol. This sol encompasses the surface-enhanced Raman nanoparticle described in the first aspect and demonstrates the ability to rapidly, easily, and accurately detect fentanyls at a low cost and with high sensitivity. Moreover, the detection is non-destructive and extends to multiple fentanyl types, showcasing exceptional technical efficacy.

In a third aspect, the present invention provides a detection method for fentanyls, wherein the detection method adopts the surface-enhanced Raman nanoparticle sol according to the second aspect for detection, preferably adds an agglomerant for detection, and more preferably adopts potassium iodide as the agglomerant for detection. The detection method can detect multiple types of fentanyls quickly, easily, with high accuracy and high sensitivity, at low cost, and non-destructively, and has excellent technical effects.

In order to solve the technical problem described above, the present invention provides the following technical solutions.

In a first aspect, the present invention provides a surface-enhanced Raman nanoparticle.

A surface-enhanced Raman nanoparticle, wherein the surface-enhanced Raman nanoparticle is a noble metal nanoparticle, the noble metal nanoparticle has a particle size of 20 nm to 200 nm, and the noble metal in the noble metal nanoparticle comprises gold or silver; the noble metal nanoparticle is one of a gold nanosphere, a gold nanocake, a gold nanorod, a gold core-shell structure, a silver nanosphere, and a silver cubic particle.

In some preferred embodiments, the noble metal nanoparticle is a gold nanocake.

In some more preferred embodiments, the noble metal nanoparticle is a gold nanocake having a diameter of 40 nm to 100 nm and a thickness of 15 nm to 35 nm. In some more preferred embodiments, the noble metal nanoparticle is a gold nanocake having a diameter of 60 nm to 90 nm and a thickness of 20 nm to 35 nm.

In some most preferred embodiments, the noble metal nanoparticle is a gold nanocake having a diameter of 80 nm±10 nm and a thickness of 30 nm±5 nm.

In a second aspect, the present invention provides a surface-enhanced Raman nanoparticle sol.

A surface-enhanced Raman nanoparticle sol, comprising the surface-enhanced Raman nanoparticle according to the first aspect and a solvent.

In some embodiments, the solvent comprises at least one of water, ethanol, and methanol.

In some embodiments, the surface-enhanced Raman nanoparticle has a structure of a gold nanocake.

In some embodiments, the surface-enhanced Raman nanoparticle sol is prepared by a method comprising: mixing a noble metal precursor solution with a protectant solution, and then adding ethanol (to adjust the morphology of the particles); after a reaction, obtaining a noble metal nanoparticle solution, centrifuging to obtain precipitate 1, washing the precipitate 1, centrifuging to obtain precipitate 2, and adding water to the precipitate 2 for mixing to obtain the surface-enhanced Raman nanoparticle sol.

In some embodiments, the noble metal precursor in the noble metal precursor solution comprises tetrachloroauric acid (HAuCl4).

In some embodiments, the noble metal precursor solution is heated to 90° C. to 105° C. before being mixed with the protectant solution. In some embodiments, the noble metal precursor solution is heated at 90° C. to 105° C. for 20 min to 35 min before being mixed with the protectant solution. The morphology of the particles may be influenced by the temperature. The heating operation by adopting the temperature provided by the present invention is beneficial to obtaining excellent particle morphology.

In some embodiments, the protectant in the protectant solution comprises at least one selected from sodium citrate, polyvinylpyrrolidone (PVP), cetyltrimethylammonium bromide (CTAB), cetyltrimethylammonium chloride (CTAC), and cetyltrimethylammonium iodide (CTAI).

In some embodiments, the noble metal precursor solution and the protectant solution are mixed within 2 seconds.

In some embodiments, the ethanol has an adding amount of 10 μL to 100 μL. In some embodiments, the ethanol has an adding amount of 10 μL, 20 μL, 30 μL, 40 μL, 50 L, 60 μL, 70 μL, 80 μL, 90 μL, or 100 μL.

In some embodiments, the reaction has a reaction temperature of 90° C. to 105° C. In some embodiments, the reaction has a reaction temperature of 90° C., 91° C., 92° C., 93° C., 94° C., 95° C., 96° C., 97° C., 98° C., 99° C., 100° C., 101° C., 102° C., 103° C., 104° C., or 105° C. In some embodiments, the reaction has a reaction temperature of 100° C.

In some embodiments, the reaction has a reaction time of 20 min to 60 min. In some embodiments, the reaction has a reaction time of 20 min, 25 min, 30 min, 35 min, 40 min, 45 min, 50 min, 55 min, or 60 min. In some embodiments, the reaction has a reaction time of 25 min to 35 min. In some embodiments, the reaction has a reaction time of 30 min.

In some embodiments, the solvent of the noble metal precursor solution comprises water.

In some embodiments, a feeding mass ratio of the noble metal precursor in the noble metal precursor solution to the protectant in the protectant solution is 10.0:3.0 to 10.0:1.0. In some embodiments, a feeding mass ratio of the noble metal precursor in the noble metal precursor solution to the protectant in the protectant solution is 10.0:3.0, 10.0:2.5, 10.0:2.0, 10.0:1.5, or 10.0:1.0. In some embodiments, a feeding mass ratio of the noble metal precursor in the noble metal precursor solution to the protectant in the protectant solution is 10.0:3.0.

In some embodiments, the noble metal precursor in the noble metal precursor solution has a concentration of 0.001 wt % to 1.000 wt %. In some embodiments, the noble metal precursor in the noble metal precursor solution has a concentration of 0.001 wt %, 0.005 wt %, 0.010 wt %, 0.020 wt %, 0.030 wt %, 0.040 wt %, 0.050 wt %, 0.060 wt %, 0.070 wt %, 0.080 wt %, 0.090 wt %, 0.100 wt %, 0.200 wt %, 0.300 wt %, 0.400 wt %, 0.500 wt %, or 1.000 wt %. In some embodiments, the noble metal precursor in the noble metal precursor solution has a concentration of 0.010 wt % to 0.050 wt %. In some embodiments, the noble metal precursor in the noble metal precursor solution has a concentration of 0.050 wt %.

In some embodiments, the protectant in the protectant solution has a concentration of 0.5 wt % to 2.0 wt %. In some embodiments, the protectant in the protectant solution has a concentration of 0.5 wt %, 0.6 wt %, 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, or 2.0 wt %. In some embodiments, the protectant in the protectant solution has a concentration of 0.8 wt % to 1.2 wt %. In some embodiments, the protectant in the protectant solution has a concentration of 1.0 wt %.

In some embodiments, the solvent of the protectant solution comprises water.

In some embodiments, the washing is performed by adopting at least one of ethanol, methanol, and water. In some embodiments, the washing is performed 1-5 times. In some embodiments, the washing is performed 1, 2, 3, 4, or 5 times.

In some embodiments, the noble metal nanoparticle in the surface-enhanced Raman nanoparticle sol has a concentration of 6×10¹¹ particles/mL to 6×10¹³ particles/mL. In some embodiments, the noble metal nanoparticle in the surface-enhanced Raman nanoparticle sol has a concentration of 6×10¹² particles/mL.

In some embodiments, the surface-enhanced Raman nanoparticle has a structure of a gold nanocake, and the surface-enhanced Raman nanoparticle sol is prepared by a method comprising: heating the noble metal precursor solution to 90° C. to 105° C. or 100° C. and heating for 20 min to 35 min; mixing the noble metal precursor solution with the protectant solution; mixing the noble metal precursor solution and the protectant solution within 2 seconds, then adding ethanol (to adjust the morphology of the particles), wherein the ethanol has an adding amount of 10 μL, reacting at 90° C. to 105° C. for 20 min to 60 min, cooling to room temperature, obtaining a noble metal nanoparticle solution, centrifuging to obtain precipitate 1, washing the precipitate 1, centrifuging to obtain precipitate 2, and adding water to the precipitate 2 for mixing to obtain the surface-enhanced Raman nanoparticle sol; the noble metal precursor in the noble metal precursor solution comprises tetrachloroauric acid (HAuCl4); the noble metal precursor in the noble metal precursor solution has a concentration of 0.05 wt %; the protectant in the protectant solution has a concentration of 1.0 wt %; the solvent of the noble metal precursor solution comprises water; the solvent of the protectant solution comprises water; the washing is performed 1-5 times; the noble metal nanoparticle in the surface-enhanced Raman nanoparticle sol has a concentration of 6×10¹¹ particles/mL to 6×10¹³ particles/mL.

In a third aspect, the present invention provides a detection method for fentanyls.

A detection method for fentanyls, comprising the following steps:
(1) extraction: taking a liquid sample to be detected or wiping a solid sample to be detected by using a wiping solvent;
(2) Raman spectrum detection: transferring the liquid sample to be detected or the wiping solvent after wiping into the surface-enhanced Raman nanoparticle sol according to the second aspect, and detecting by using a Raman spectrum with a laser wavelength of 785 nm, 633 nm, 532 nm and/or 1064 nm to obtain Raman frequency shift spectrum data of the liquid sample to be detected;
(3) result judgment: wherein the liquid sample to be detected that has a peak at least at 1, 2, 3, or 4 positions at wave numbers of 832±6 cm-1, 1000±6 cm-1, 1030±6 cm-1, and 1200±6 cm-1 in the Raman frequency shift spectrum data comprises fentanyls; or the solid sample to be detected or the liquid sample to be detected that has a peak at least at 1, 2, or 3 positions at wave numbers of 1000±6 cm-1, 1030±6 cm-1, and 1200±6 cm-1 in the Raman frequency shift spectrum data comprises fentanyls; the solid sample to be detected or the liquid sample to be detected that has no peak at wave numbers of 832±6 cm-1, 1000±6 cm-1, 1030±6 cm-1, and 1200±6 cm-1 in the Raman frequency shift spectrum data does not comprise fentanyls.

In some embodiments, the Raman spectrum detection in step (2) further comprises adding an agglomerant solution after transferring the liquid sample to be detected or the wiping solvent after wiping into the surface-enhanced Raman nanoparticle sol according to the second aspect and before performing the detection by using Raman spectrum.

In some embodiments, the agglomerant in the agglomerant solution comprises at least one of KCl, KBr, KI, MgSO4, MgCl2, NaBr, and NaCl. In some preferred embodiments, the agglomerant in the agglomerant solution is KI.

In some embodiments, the agglomerant in the agglomerant solution has a concentration of 0.01 mol/L to 5.00 mol/L. In some embodiments, the agglomerant in the agglomerant solution has a concentration of 0.01 mol/L, 0.05 mol/L, 0.10 mol/L, 0.50 mol/L, 0.60 mol/L, 0.70 mol/L, 0.80 mol/L, 0.90 mol/L, 1.00 mol/L, 1.10 mol/L, 1.20 mol/L, 1.30 mol/L, 1.40 mol/L, 1.50 mol/L, 2.00 mol/L, 2.50 mol/L, 3.00 mol/L, 3.50 mol/L, 4.00 mol/L, 4.50 mol/L, or 5.00 mol/L. In some embodiments, the agglomerant in the agglomerant solution has a concentration of 1.00 mol/L.

In some embodiments, the agglomerant solution adopts at least one of water, methanol, and ethanol as a solvent.

In some embodiments, the excitation power of the Raman spectrum is 10 mW to 500 mW. In some embodiments, the excitation power of the Raman spectrum is 10 mW, 50 mW, 100 mW, 150 mW, 200 mW, 250 mW, 300 mW, 350 mW, 400 mW, 450 mW, or 500 mW.

In some embodiments, the wiping solvent is at least one of methanol, acetone, ethanol, or diethyl ether.

In some embodiments, the wiping a solid sample to be detected by using a wiping solvent comprises wiping the solid sample to be detected by using a cotton swab after the cotton swab is wetted with the wiping solvent.

In some embodiments, the wiping solvent has a volume of 0.1 mL to 1.0 mL. In some embodiments, the wiping solvent has a volume of 0.1 mL, 0.2 mL, 0.3 mL, 0.4 mL, 0.5 mL, 0.6 mL, 0.7 mL, 0.8 mL, 0.9 mL, or 1.0 mL.

In some embodiments, the wiping is performed 1-5 times. In some embodiments, the wiping is performed 1, 2, 3, 4, or 5 times.

In some embodiments, the wiping is performed for 1 second to 10 seconds. In some embodiments, the wiping is performed for 1 second, 2 seconds, 3 seconds, 4 seconds, 5 seconds, 6 seconds, 7 seconds, 8 seconds, 9 seconds, or 10 seconds.

In some embodiments, the transferring comprises transferring by adopting infiltration, dripping, or pressing.

In some embodiments, the volume ratio of the liquid sample to be detected or the wiping solvent to the surface-enhanced Raman nanoparticle sol is 1:10 to 1:1. In some embodiments, the volume ratio of the liquid sample to be detected or the wiping solvent to the surface-enhanced Raman nanoparticle sol is 1:10, 1:9, 1:8, 1:7, 1:6, 1:5, 1:4, 1:3, 1:2, or 1:1. In some embodiments, the volume ratio of the liquid sample to be detected or the wiping solvent to the surface-enhanced Raman nanoparticle sol is 1:2.

In some embodiments, the volume ratio of the agglomerant solution to the surface-enhanced Raman nanoparticle sol is 1:10 to 1:1. In some embodiments, the volume ratio of the agglomerant solution to the surface-enhanced Raman nanoparticle sol is 1:10, 1:9, 1:8, 1:7, 1:6, 1:5, 1:4, 1:3, 1:2, or 1:1. In some embodiments, the volume ratio of the agglomerant solution to the surface-enhanced Raman nanoparticle sol is 1:4.

In some embodiments, the solid sample to be detected has a material comprising metal, ceramic, glass, plastic, stone, wood, or skin.

In some embodiments, the solid sample to be detected includes mail, a package, a packing box, a packing bag, an electric appliance, furniture, a container, or skin.

In some embodiments, the Raman spectrum comprises performing detection by using a portable Raman spectrometer or a hand-held Raman spectrometer.

In a fourth aspect, the present invention provides the surface-enhanced Raman nanoparticle according to the first aspect, the surface-enhanced Raman nanoparticle sol according to the second aspect, or use of the detection method according to the third aspect.

Use of the surface-enhanced Raman nanoparticle according to the first aspect, the surface-enhanced Raman nanoparticle sol according to the second aspect, or the detection method according to the third aspect for the detection of fentanyls, for the rapid detection of a trace amount of fentanyls, for the rapid detection of a trace amount of fentanyls on the surface of mail or a package, or for the rapid non-destructive detection of a trace amount of fentanyls on the surface of mail or a package.

In some embodiments, the fentanyls comprises at least one of 4-fluorobutyrylfentanyl, 4-fluoroisobutyrylfentanyl, butyrylfentanyl, isobutyrylfentanyl or a salt thereof, furanylfentanyl or a salt thereof, valerylfentanyl or a salt thereof, β-hydroxythiofentanyl, cis-3-methylfentanyl or a salt thereof, ocfentanil, p-fluorofentanyl, sufentanil citrate, acetylfentanyl, β-hydroxy-3-methylfentanyl, 4-anilino-N-phenylethylpiperidine, remifentanil or a salt thereof, α-methylfentanyl or a salt thereof, N-phenylethyl-4-piperidone, carfentanil, β-hydroxyfentanyl, 3-methylthiofentanyl or a salt thereof, alfentanil, fentanyl, acetyl-alpha-methylfentanyl, acrylfentanyl or a salt thereof, thiofentanyl or a salt thereof, alpha-methylthiofentanyl or a salt thereof, tetrahydrofuranylfentanyl, 2-thenoylfentanyl or a salt thereof, chloroacetylfentanyl or a salt thereof, benzoylfentanyl or a salt thereof, (2-fluorobenzoyl)fentanyl or a salt thereof, (3-fluorobenzoyl)fentanyl or a salt thereof, (2-chlorobenzoyl)fentanyl or a salt thereof, (4-fluorobenzoyl)fentanyl or a salt thereof, p-chlorofuranylformylfentanyl or a salt thereof, β-chloromethoxyacetylfentanyl or a salt thereof, p-chlorothienylformylfentanyl or a salt thereof, p-chlorobenzoylfentanyl or a salt thereof, p-chlorocyclopropylformylfentanyl or a salt thereof, p-chloroacetylfentanyl or a salt thereof, cyclopentylformylfentanyl or a salt thereof, cyclobutylformylfentanyl or a salt thereof, heptanoylfentanyl or a salt thereof, ethoxyacetylfentanyl or a salt thereof, phenylpropanoylfentanyl or a salt thereof, butyryl-alpha-methylfentanyl or a salt thereof, cyclobutylformylfentanyl or a salt thereof, isovalerylfentanyl or a salt thereof, N-benzylbutyrylfentanyl or a salt thereof, N-benzylcyclopropylfentanyl or a salt thereof, N-benzylpentanoylfentanyl or a salt thereof, N-benzylacetylfentanyl or a salt thereof, N-benzylhexanoylfentanyl or a salt thereof, alpha-methylfentanyl or a salt thereof, beta-hydroxy-3-methylfentanyl or a salt thereof, beta-hydroxyfentanyl or a salt thereof, beta-hydroxyisobutyrylfentanyl or a salt thereof, beta-hydroxyvalerylfentanyl or a salt thereof, thiofentanyl or a salt thereof, ethoxyacetylfentanyl or a salt thereof, cyclopentylformylfentanyl or a salt thereof, p-methylbutyrylfentanyl or a salt thereof, o-methylfentanyl or a salt thereof, p-methoxyacryloylfentanyl or a salt thereof, N-benzylfentanyl or a salt thereof, N-benzyl-p-fluorofentanyl or a salt thereof, norcarfentanil or a salt thereof, N-(4-methylphenylethyl)-isobutyrylfentanyl or a salt thereof, phenylpropanoylfentanyl or a salt thereof, heptanoylfentanyl or a salt thereof, 2-thienylformylfentanyl or a salt thereof, chloroacetylfentanyl or a salt thereof, benzoylfentanyl or a salt thereof, cyclobutylformylfentanyl or a salt thereof, (2-fluorobenzoyl)fentanyl or a salt thereof, (3-fluorobenzoyl)fentanyl or a salt thereof, isovaleroylfentanyl or a salt thereof, (2-chlorobenzoyl)fentanyl or a salt thereof, (4-fluorobenzoyl)fentanyl or a salt thereof, p-fluoroacetylfentanyl or a salt thereof, p-fluorotetrahydrofuranylformylfentanyl or a salt thereof, p-fluorobenzoylfentanyl or a salt thereof, p-fluorothienylformylfentanyl or a salt thereof, p-fluorofuranylformylfentanyl or a salt thereof, p-fluorocyclopentanoylformylfentanyl or a salt thereof, p-fluoropentanoylfentanyl or a salt thereof, o-fluorofentanyl or a salt thereof, o-fluoroacroloylfentanyl or a salt thereof, o-fluoroacetylfentanyl or a salt thereof, m-fluorofentanyl or a salt thereof, m-fluoromethoxyacetylfentanyl or a salt thereof, m-fluoroisobutyrylfentanyl or a salt thereof, m-fluoroacetylfentanyl or a salt thereof, m-fluorofuranylfentanyl or a salt thereof, m-fluorobenzoylfentanyl or a salt thereof, p-chlorofentanyl or a salt thereof, p-chlorobutyrylfentanyl or a salt thereof, p-chlorofuranylformylfentanyl or a salt thereof, p-chloromethoxyacetylfentanyl or a salt thereof, p-chlorothienylformylfentanyl or a salt thereof, p-chlorobenzoylfentanyl or a salt thereof, p-chlorocyclopropylformylfentanyl or a salt thereof, p-chloroacetylfentanyl or a salt thereof, p-methylfentanyl or a salt thereof, p-methyl-(4-fluorobenzoyl)fentanyl or a salt thereof, p-methylcyclopentylformylfentanyl or a salt thereof, p-methylcyclohexylformylfentanyl or a salt thereof, p-methyl-tert-butylformylfentanyl or a salt thereof, p-methylcyclopropylformylfentanyl or a salt thereof, p-methylmethoxyacetylfentanyl or a salt thereof, p-methylthienylformylfentanyl or a salt thereof, p-methylfuranylformylfentanyl or a salt thereof, p-methylbenzoylfentanyl or a salt thereof, p-methylethoxyacetylfentanyl or a salt thereof, p-methyltetrahydrofuranylformylfentanyl or a salt thereof, p-methyl-(4-chlorobenzoyl)fentanyl or a salt thereof, o-methylfuranylformylfentanyl or a salt thereof, o-methylcyclohexylformylfentanyl or a salt thereof, o-methylbutyrylfentanyl or a salt thereof, o-methylbenzoylfentanyl or a salt thereof, o-methyl-(4-fluorobenzoyl)fentanyl or a salt thereof, o-methylthienylformylfentanyl or a salt thereof, o-methylcyclopentylformylfentanyl or a salt thereof, benzoylalpha-methylfentanyl or a salt thereof, hexanoylalpha-methylfentanyl or a salt thereof, p-methoxytetrahydrofuranylfentanyl or a salt thereof, p-methoxyisobutyrylfentanyl or a salt thereof, p-methoxy-2-methoxyacetylfentanyl or a salt thereof, p-methoxyhexanoylfentanyl or a salt thereof, thioacetylfentanyl or a salt thereof, N-benzylacetylfentanyl or a salt thereof, N-benzylbutyrylfentanyl or a salt thereof, N-benzyl-(4-chlorobenzoyl)fentanyl or a salt thereof, N-benzylcyclopropylfentanyl or a salt thereof, N-benzylcyclopentylfentanyl or a salt thereof, N-benzylvalerylfentanyl or a salt thereof, N-benzylfuranylfentanyl or a salt thereof, N-benzylhexanoylfentanyl or a salt thereof, N-benzyl-p-fluorocyclopentylformylfentanyl or a salt thereof, N-benzyl-p-fluoro-cyclohexylformylfentanyl or a salt thereof, N-benzyl-p-fluoro-furanylformylfentanyl or a salt thereof, N-benzyl-p-fluoro-(3-fluorobenzoyl)fentanyl or a salt thereof, N-benzyl-p-fluoro-acetylfentanyl or a salt thereof, N-benzyl-p-fluoro-thienylformylfentanyl or a salt thereof, N-benzyl-p-fluoro-methoxyacetylfentanyl or a salt thereof, N-benzyl-p-fluoro-isobutyrylfentanyl or a salt thereof, N-benzyl-p-fluoro-butyrylfentanyl or a salt thereof, N-benzyl-p-fluoro-(4-chlorobenzoyl)fentanyl or a salt thereof, N-methylbenzoylfentanyl or a salt thereof, N-methyl-(4-chlorobenzoyl)fentanyl or a salt thereof, N-methyl-(2-fluorobenzoyl)fentanyl or a salt thereof, N-(4-methylphenylethyl)-benzoylfentanyl or a salt thereof, N-(4-methylphenylethyl)fentanyl or a salt thereof, N-(4-methylphenylethyl)-isovalerylfentanyl or a salt thereof, N-(4-methylphenylethyl)-n-butyrylfentanyl or a salt thereof, N-cyclopropylformylcyclopropylfentanyl or a salt thereof, p-fluorofentanyl or a salt thereof, N-(4-methylphenylethyl)-(4-chlorobenzoyl)fentanyl or a salt thereof, N-(4-methylphenylethyl)-thienylformylfentanyl or a salt thereof, N-(4-nitrophenylethyl)fentanyl or a salt thereof, valerylalpha-methylfentanyl or a salt thereof, N-benzylisobutyrylfentanyl or a salt thereof, N-(4-methylphenylethyl)-cyclohexylformylfentanyl or a salt thereof, N-(4-chlorophenylethyl)fentanyl or a salt thereof, N-(4-methylphenylethyl)-methoxyacetylfentanyl or a salt thereof, N-(4-methylphenylethyl)-acetylfentanyl or a salt thereof, o-methoxybutyrylfentanyl or a salt thereof, o-methoxyvalerylfentanyl or a salt thereof, 3-methylthioacetylfentanyl or a salt thereof, butyrylalpha-methylfentanyl or a salt thereof, betahydroxy-3-methylbutyrylfentanyl or a salt thereof, and 3-methylthiobutyrylfentanyl or a salt thereof.

In some embodiments, the fentanyls comprises at least one of fentanyl, α-methyl-fentanyl hydrochloride, β-hydroxy-fentanyl hydrochloride, (±)cis-3-methylfentanyl, 3-methylthiofentanyl, p-fluorofentanyl, remifentanil hydrochloride, sufentanil citrate, acetylfentanyl, butyrylfentanyl, (±)-β-hydroxythiofentanyl hydrochloride, and 4-fluorobutyrylfentanyl.

### Beneficial Effects

Compared with the prior art, one embodiment of the present invention at least has one of the following beneficial technical effects:

1. Direct Raman detection is hindered by the low concentration of fentanyls present in mail, packages, and similar materials. Furthermore, conventional detection methods face challenges due to multiple interferents, leading to the difficulty of avoiding false identifications. In this invention, we propose a novel approach. Following the rapid extraction and transfer of fentanyls from mail and packages, the drug molecules are introduced into a surface-enhanced Raman nanoparticle sol developed in this work. Detection is then carried out using a portable Raman spectrometer or a handheld Raman spectrometer, enabling ultra-sensitive and non-destructive identification of fentanyls in mail and packages. The detection limit of the detection method of the present invention for fentanyls is as low as 1 μg/L.

2. The present invention provides an ultra-sensitive and non-destructive detection for fentanyls in mail and packages. A spectrogram is obtained by performing surface-enhanced Raman detection on samples such as mail and packages, and whether the mail, packages, and the like contain fentanyls or not can be quickly obtained from the spectrogram, which is very suitable for quickly detecting fentanyls in the mail and packages by customs. Conventional methods require minutes per sample, whereas the technological solution presented in this invention achieves a processing time of seconds per sample.

3. The method proposed by the present invention is straightforward to operate, cost-effective, and rapid. It does not require a large-scale, precise instrument, enabling field-portable detection of fentanyls in mail and packages. The proficiency requirements for operators are minimal, allowing ordinary individuals to quickly master and perform the detection process, thereby significantly alleviating operational challenges. The procedure involves merely wiping with a cotton swab, ensuring simplicity and convenience and resulting in substantial savings in labor costs.

4. The present invention can be applied to the field of public safety detection, in particular to application scenes such as customs and a port. A quick and effective screening means is provided, which can improve the detection efficiency, reduces the detection cost, is hopeful to be popularized to practical application for rapidly and non-destructively detecting fentanyls, has very large application prospect, and is also a key area supported by national security. The current technical maturity needs to be improved. The amount of mail and packages is very large, and the propagation and circulation of drugs are secretly disguised. Trace amount rapid analysis has very good application prospect in public security detection.

5. Compared with other shapes of surface-enhanced Raman nanoparticles, the cake-shaped nanoparticle is preferred in the present invention, and the cake-shaped gold nanoparticle is even more preferable. This preference is beneficial for enhancing the detection sensitivity of the surface-enhanced Raman nanoparticle or the surface-enhanced Raman nanoparticle sol described in the present invention for fentanyls.

6. Compared with surface-enhanced Raman nanoparticles with other sizes, the gold nanocake having a diameter of 80 nm and a thickness of 30 nm is preferably adopted as the surface-enhanced Raman nanoparticle, which is more beneficial for improving the detection sensitivity of the surface-enhanced Raman nanoparticle or the surface-enhanced Raman nanoparticle sol described in the present invention on fentanyls.

7. According to the present invention, the agglomerant solution is added during the detection of fentanyls, which is beneficial for improving the sensitivity of the detection method.

8. The agglomerant in the agglomerant solution described in the present invention is preferably selected from at least one of KCl, KBr, KI, MgSO4, MgCl2, NaBr, and NaCl, which is beneficial for avoiding the interference of the agglomerant on the detection peak of fentanyls; the agglomerant in the agglomerant solution is more preferably KI, which is more beneficial for improving the sensitivity of the detection method.

### BRIEF DESCRIPTION OF DRAWINGS

FIG. 1 is the measurements of different types of surface-enhanced Raman nanoparticle sol obtained in Examples 1 and 2 on a fentanyl standard solution at the same concentration and volume.

FIG. 2 is the ability test of different types of agglomerants in Example 4 to enhance results.

FIG. 3 is the interference test of different types of agglomerants in Example 4 on the test itself.

FIG. 4 is the scanning electron microscope characterization on the surface-enhanced Raman nanoparticle in Example 5 after added with different salt solutions as the agglomerant.

FIG. 5 is the stability test for the detection on fentanyl using KI as an agglomerant in Example 6.

FIG. 6 is the detection of the gold cake nanoparticle in Example 7 on different types of fentanyl standard substances.

FIG. 7 is the detection of the gold cake nanoparticle in Example 7 on different types of fentanyl standard substances.

### DEFINITIONS OF TERMS

In the present invention, “room temperature” presents ambient temperature, and may be 20° C. to 30° C.; in some embodiments, the temperature is 22° C. to 28° C.; in some embodiments, the temperature is 24° C. to 26° C.; and in some embodiments, the temperature is 25° C.

In the context of the present invention, all numbers disclosed herein are approximate values, whether or not the word “about” or “approximately” is used. Based on the disclosed numbers, it is possible that the numerical value of each number may have a difference of ±10% or less or a reasonable difference considered by those skilled in the art, such as by ±1%, ±2%, ±3%, ±4%, or ±5%.

The term “optional” or “optionally” means that the subsequently described event or case may or may not occur. For example, “optional surfactant” means that the surfactant may or may not be present.

The term “weight percentage” or “percentage by weight” or “wt %” is defined as follows: The weight of an individual component in a composition is divided by the total weight of all components in the composition and multiplied by 100.

The term “and/or” should be understood to refer to any one of the options or any two or more of the options.

The term “wt %” means mass percentage.

The term “Raman frequency shift value” of a substance is the wavenumber at which a peak appears in a Raman spectrum and is referred to as the Raman frequency shift value of the substance.

In the present invention, the “particle size” of non-spherical particles means the width between two points having the farthest width in the non-spherical particle.

In the specification, terms such as “one embodiment”, “some embodiments”, “examples”, “a specific example”, or “some examples”, mean that a particular feature, structure, material, or characteristic described in reference to the embodiment or example is included in at least one embodiment or example of the present invention. In this specification, the schematic descriptions of the terms described above do not necessarily refer to the same embodiment or example. Moreover, the specific features, materials, structures, and characteristics described may be combined in any one or more embodiments or examples in an appropriate manner. Moreover, various embodiments or examples and features of various embodiments or examples described in this specification can be combined by one skilled in the art to the extent that they do not contradict each other.

### DETAILED DESCRIPTION

In order to make the technical solutions of the present invention better understood by those skilled in the art, some non-limiting examples are further disclosed below to further explain the present invention in detail.

The reagents used in the present invention are either commercially available or can be prepared by the methods described herein.

The detection limit described in the present invention is the detection limit concentration of fentanyls in a liquid sample to be detected or a wiping solvent obtained after a solid sample to be detected is wiped by using the wiping solvent.

### Example 1: Preparation of Surface-Enhanced Raman Nanoparticle Sol of Gold

### Nanocake

1. Synthesis of a gold nanocake: 200 mL of an aqueous HAuCl4 solution having a mass fraction of 0.050% was continuously heated and stirred, and boiled (100° C.) for 30 min. Then 3 mL of an aqueous sodium citrate solution having a mass fraction of 1.0% was rapidly injected (added completely within 2 seconds), and 10 μL of ethanol was added. Then the mixture was boiled (100° C.) and refluxed for 30 min, and then cooled to room temperature to obtain an aqueous solution of metal nanoparticles.

2. Cleaning and enriching of particles: The aqueous solution of the metal nanoparticle obtained in step 1 was centrifuged, the supernatant was removed to obtain precipitate 1, water was added to the precipitate 1 for mixing, and the mixture was centrifuged to obtain precipitate 2. The precipitate 2 was the cleaned metal nanoparticle (the purpose of cleaning was to remove redundant sodium citrate as a protectant on the particle surface, and the synthesized metal nanoparticle needed to be cleaned with water, ethanol, or methanol, preferably water). Then a proper amount of deionized water was added to the cleaned metal nanoparticle to ensure that the concentration of the metal nanoparticle is 6×10¹² particles/mL (which may be 6×10¹¹ particles/mL to 6×10¹³ particles/mL) to obtain the surface-enhanced Raman nanoparticle sol. The shape and size of the surface-enhanced Raman nanoparticle in the obtained surface-enhanced Raman nanoparticle sol were detected. The obtained surface-enhanced Raman nanoparticle was analyzed to be cake-shaped through a scanning electron microscope result, the diameter was 80 nm, and the thickness was 30 nm.

### Example 2: Detection of Fentanyls

Extraction of fentanyls: A cotton swab was wetted with ethanol (which may be at least one of methanol and ethanol, and the volume ratio of methanol to ethanol may be 1:0 or 0:1 or 1:1), and used to wipe the surface of a sample to be detected, which contained fentanyls, to extract the fentanyls, wherein the wiping was performed 2 times for a total wiping time of 5 s.

Transfer and detection of fentanyls: Known fentanyl standard samples were detected directly for testing the sensitivity of the surface-enhanced Raman nanoparticle to fentanyl samples. In a practical application embodiment, after the fentanyls were extracted by using a cotton swab, the fentanyls were soaked into the surface-enhanced Raman nanoparticle sol obtained in step (1), so that the fentanyls were transferred into the surface-enhanced Raman nanoparticle sol, and the total soaking time was 10 s; then the detection was performed by adopting a Raman spectrometer; detection conditions: The laser wavelength was 785 nm, and the excitation power was 100 mW; the integration time was automatic, the detection and identification process of the instrument did not exceed 20 s usually, and the number of integration was 1. The Raman instrument model was SHINS-P785V, Xiamen SHINs Technology Co, Ltd.

### Example 3: Performance Investigation of Different Surface-Enhanced Raman

### Nanoparticle Sol for Detecting Fentanyl

### 1. Preparation of Different Types of Surface-Enhanced Raman Nanoparticle Sol

1) Preparation of Au ball (particle size 55 nm) sol: 200 mL of an aqueous HAuCl4 solution having a mass fraction of 0.010% was continuously heated and stirred, and boiled (100° C.) for 30 min. Then 1.5 mL of a sodium citrate solution having a mass fraction of 1.0% was rapidly injected (added completely within 2 seconds). Then the mixture was boiled and refluxed for 30 min, and then cooled to room temperature to obtain an aqueous solution of metal nanoparticles. The aqueous solution of the metal nanoparticle was centrifuged, the supernatant was removed to obtain precipitate 1, water was added to the precipitate 1 for mixing, and the mixture was centrifuged to obtain precipitate 2. The precipitate 2 was the cleaned metal nanoparticle. Then a proper amount of deionized water was added to the cleaned metal nanoparticle to ensure that the concentration of the metal nanoparticle was 6×10¹² particles/mL to obtain the surface-enhanced Raman nanoparticle sol. The shape and size of the surface-enhanced Raman nanoparticle in the obtained surface-enhanced Raman nanoparticle sol were detected. The obtained surface-enhanced Raman nanoparticle was analyzed to be spherical through a scanning electron microscope result. The particle size was 55 nm. The prepared surface-enhanced Raman nanoparticle sol was named as Au ball (particle size 55 nm) sol.

2) Preparation of Au ball (particle size 30 nm) sol: 200 mL of an aqueous HAuCl4 solution having a mass fraction of 0.010% was continuously heated and stirred, and boiled (100° C.) for 30 min. Then 3 mL of an aqueous sodium citrate solution having a mass fraction of 1.0% was added within 2 seconds. Then the mixture was boiled and refluxed for 30 min, and then cooled to room temperature to obtain an aqueous solution of metal nanoparticles. The aqueous solution of the metal nanoparticle was centrifuged, the supernatant was removed to obtain precipitate 1, water was added to the precipitate 1 for mixing, and the mixture was centrifuged to obtain precipitate 2. The precipitate 2 was the cleaned metal nanoparticle. Then a proper amount of deionized water was added to the cleaned metal nanoparticle to ensure that the concentration of the metal nanoparticle was 6×10¹² particles/mL to obtain the surface-enhanced Raman nanoparticle sol. The shape and size of the surface-enhanced Raman nanoparticle in the obtained surface-enhanced Raman nanoparticle sol were detected. The obtained surface-enhanced Raman nanoparticle was analyzed to be spherical through a scanning electron microscope result. The particle size was 30 nm. The prepared surface-enhanced Raman nanoparticle sol was named as Au ball (particle size 30 nm) sol.

3) Preparation of Ag ball (different particle sizes) sol (100 nm as an example):
(1) firstly, 10 mL of 45 nm Au seeds were placed in a 100 mL flask, and then 58 mL of ultrapure water was added;
(2) then 0.84 mL of an aqueous sodium citrate solution having a mass fraction of 1 wt % and 0.84 mL of an aqueous ascorbic acid solution having a mass fraction of 1 wt % were separately transferred into the Au seed solution, and the mixture was uniformly stirred for 5 min;
(3) finally, 9.6 mL (1.344 mL of 20 mM AgNO3 was diluted to 9.6 mL) of an AgNO3 solution was added into the round-bottom flask at a certain dropping rate by pushing a syringe;
(4) the dropwise addition was completed in about 45 minutes, followed by centrifugation and characterization. The amounts of each substance required for the synthesis of different sizes of Ag particles are shown in the table below:

| | | | Addition | | |
|---|---|---|---|---|---|
| | | | amount of 1 | Addition | |
| | | | wt % | amount of 1 | |
| | | Addition | aqueous | wt % | Addition |
| | Addition | amount of | sodium | aqueous | amount of |
| Ag ball | amount of | ultrapure | citrate | ascorbic | 20 mM |
| particle size | Au seed | water in step | solution | acid solution | AgNO3 |
| (nm) | (mL) | (1) (mL) | (mL) | (mL) | (mL) |
| 80 | 10 | 59.0 | 0.41 | 0.41 | 0.580 |
| 100 | 10 | 58.0 | 0.84 | 0.84 | 1.344 |
| 120 | 10 | 57.5 | 1.20 | 1.20 | 1.920 |
| 150 | 10 | 55.0 | 2.40 | 2.40 | 3.840 |

4) Preparation of Ag cube (particle size 80 nm) sol: A clean and dried round-bottom flask was taken. 20 mL of 1,5-pentanediol was added and incubated at 195° C. for 10 min. Then two syringes were taken for sucking an appropriate amount of an AgNO3 solution and a polyvinylpyrrolidone solution, and then the AgNO3 solution and the polyvinylpyrrolidone solution were started to be added dropwise to the thermostatic pentanediol. The solution was observed to change color from colorless to grayish green, and 80 nm Ag cubes were obtained.

5) Preparation of 55 nm Au@3 nm MOF sol: An organic ligand dimethylimidazole was added to a 55 nm Au solution to synthesize an Au @3 nm MOF structure. During the synthesis of Au@3 nm MOF, particles with different shell thicknesses could be precisely prepared by slightly adjusting the amounts of Zn ions and the added 2-methylimidazole, wherein the ratio of Zn ions to 2-methylimidazole was kept at 1:3.

6) 55 nm Au@4 nm ZrO2 sol and 55 nm Au@2 nm TiO2 sol were purchased from Xiamen SHINs Technology Co., Ltd.

2. Sensitivity investigation of different types of surface-enhanced Raman nanoparticle sol:
200 μL of different types of surface-enhanced Raman nanoparticle sol was separately added to a test tube, then 100 μL of a 0.1 mg/L fentanyl ethanol solution and 50 μL of a 1 mol/L KI solution were separately added. The mixture was mixed to obtain a sensitivity test solution, and detected by using a Raman spectrometer; the detection conditions of the Raman spectrometer were the same as Example 2.

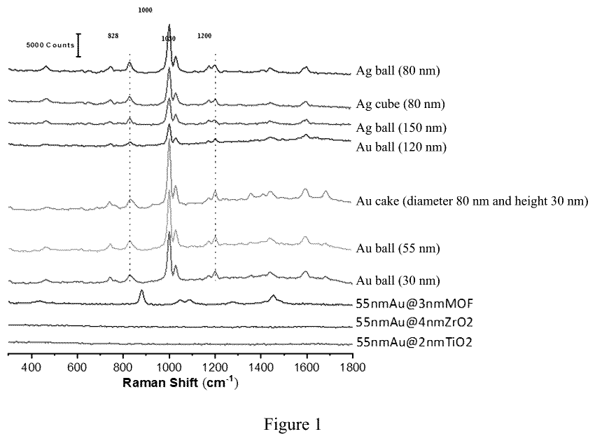

Results: FIG. 1 shows the detection of different types of surface-enhanced Raman nanoparticle sol on 100 μL of a 0.1 mg/L fentanyl standard solution. As is apparent from FIG. 1, the spectrogram of the 0.1 mg/L fentanyl ethanol solution has at least 2 peaks at 832±6 cm-1, 1000±6 cm-1, 1030±6 cm-1, and 1200±6 cm-1 shifts, wherein the gold nanocake has the strongest peak intensity at the characteristic peak of fentanyl, and therefore, the gold nanocake has the strongest surface Raman enhancement capability.

### Example 4: Optimization of Detection Method of Gold Cake Nanoparticles on

### Fentanyls

Preparation of salt solutions of different types of agglomerants: Different types of agglomerants KCl, KBr, KI, MgSO4, MgCl2, Na2SO4, NaBr, and NaCl were separately dissolved in water until the concentrations of the agglomerants were 1.00 mol/L, and salt solutions of the different types of agglomerants were separately obtained.

Detection sensitivity investigation of different types of agglomerants: 200 μL of the surface-enhanced Raman nanoparticle sol obtained in Example 1 was separately added to different test tubes, then 100 μL of a 0.1 mg/L fentanyl ethanol solution and 50 μL of 1.00 mol/L salt solutions of different types of agglomerants were separately added. The mixture was mixed, and detected by using a Raman spectrometer; the detection conditions of the Raman spectrometer were the same as Example 2.

Detection sensitivity investigation without addition of an agglomerant: 200 μL of the surface-enhanced Raman nanoparticle sol obtained in Example 1 was separately added to different test tubes, then 100 μL of a 0.1 mg/L fentanyl ethanol solution and 50 μL of water were separately added. The mixture was mixed, and detected by using a Raman spectrometer; the detection conditions of the Raman spectrometer were the same as Example 2.

Blank interference tests of different types of agglomerants: 200 μL of the surface-enhanced Raman nanoparticle sol obtained in Example 1 was separately added to different test tubes, then 100 μL of ethanol and 50 μL of 1.00 mol/L salt solutions of different types of agglomerants were separately added. The mixture was mixed, and detected by using a Raman spectrometer; the detection conditions of the Raman spectrometer were the same as Example 2.

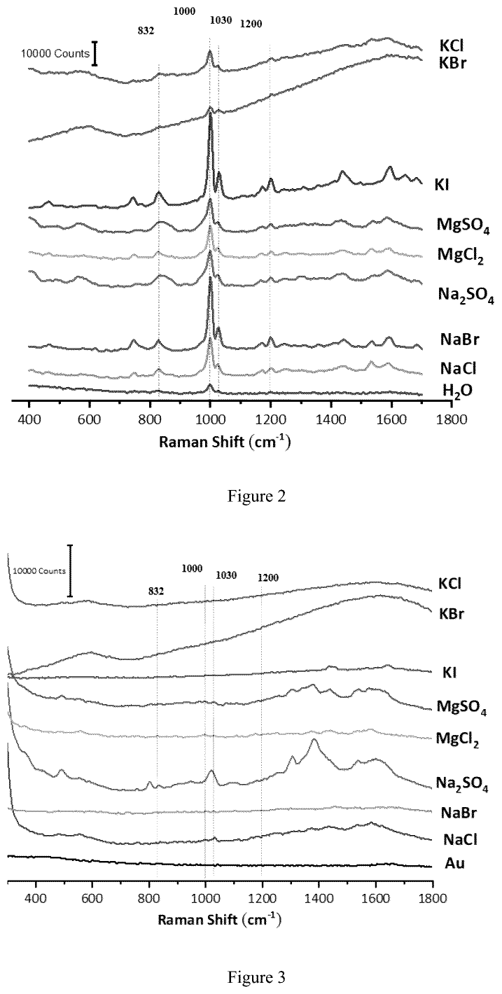

The results are shown in FIGS. 2 and 3.

FIG. 2 is the effect of adding different types of agglomerants, wherein the surface-enhanced Raman nanoparticle sol has the same volume (200 μL) and the fentanyl standard solution also has the same volume (100 μL). As is apparent from FIG. 2, when other agglomerants are not added and only water with the same volume is added, the characteristic peak signal of fentanyl is relatively weak, and after the agglomerant is added, the characteristic peak signal of fentanyl can be significantly enhanced, which shows that the agglomerant can accelerate the aggregation of particles, and a signal with higher intensity is further obtained. The spectrogram for testing the signal influence of different types of agglomerants on fentanyls has at least 2 peaks at 832±6 cm-1, 1000±6 cm-1, 1030±6 cm-1, and 1200±6 cm-1 shifts, wherein the signal intensity is strongest when KI (potassium iodide) is used as the agglomerant, and therefore KI is preferably used as the agglomerant.

FIG. 3 is the investigation on whether the addition of different types of agglomerants interferes with the detection results. Since the detection solution did not contain fentanyl standard, it is apparent from FIG. 3 that during the test, when Na2SO4 was used as the agglomerant, interference existed at 1000±6 cm-1 and 1030±6 cm-1 shifts, and interference did not exist on the detection of a blank sample by other salts.

### Example 5: Mechanism Study of Different Salt Solutions as Agglomerant

To investigate the mechanism of salt solutions of different types of agglomerants as the agglomerant, 200 μL of the surface-enhanced Raman nanoparticle sol obtained in Example 1 was added to a test tube, then 100 μL of a 0.1 mg/L fentanyl ethanol solution and 50 μL of 1.00 mol/L salt solutions of different types of agglomerants were separately added (the preparation method for the salt solutions of different types of agglomerants is described in the preparation of salt solutions of different types of agglomerants in “(1) optimization of the type of salt solutions as the agglomerant” in Example 2). The mixture was mixed, and detected by using a Raman spectrometer; the detection conditions of the Raman spectrometer were the same as Example 2.

The purpose of adding the agglomerant was to agglomerate the surface-enhanced Raman nanoparticles more quickly. The distance between particles became shorter, thereby having stronger enhancing capability. By performing electron microscope characterization on the surface-enhanced Raman nanoparticle after added with different salt solutions as the agglomerant, it was found that under the same condition, when KI was the agglomerant, the agglomeration of the particles was the most compact.

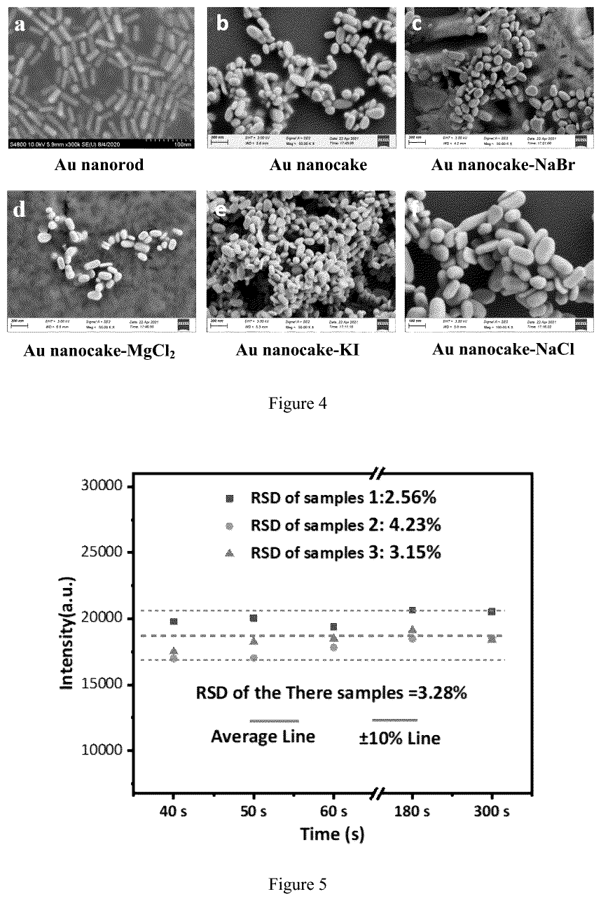

The results are shown in FIG. 4.

FIG. 4 is the scanning electron microscope characterization. FIG. 4a is the characterization of a gold nanorod. FIGS. 4b-4f are the scanning electron microscope characterization of a gold nanocake and the gold nanocake after added with different agglomerants.

### Example 6: Stability Test for Detection Using KI as Agglomerant

Stability test for detection using KI as agglomerant: To investigate the stability for the detection using KI as an agglomerant. 200 μL of the surface-enhanced Raman nanoparticle sol obtained in Example 1 was added to a test tube, then 100 μL of a 0.1 mg/L fentanyl ethanol solution and 50 μL of a 1 mol/L aqueous KI solution were separately added. The mixture was mixed, and detected by using a Raman spectrometer at different times after mixing; the detection conditions of the Raman spectrometer were the same as Example 2, and the results are shown in FIG. 5.

Analysis of results: The test signal changes over time. A stable system is the key to the detection, and therefore, the stability of the signal after addition of the agglomerant was measured. FIG. 5 is a stability performance test. The variation of the signal intensity over time at 1000±6 cm-1 shift was counted. It can be seen that in this system, the Raman signal intensity of fentanyl is substantially stable within 300 s, and the average RSD is 3.28% when measured in three groups in parallel. The results reflect the good stability of KI as an agglomerant.

### Example 7: Detection of Gold Cake Nanoparticle on Different Types of Fentanyl

### Standard Substances

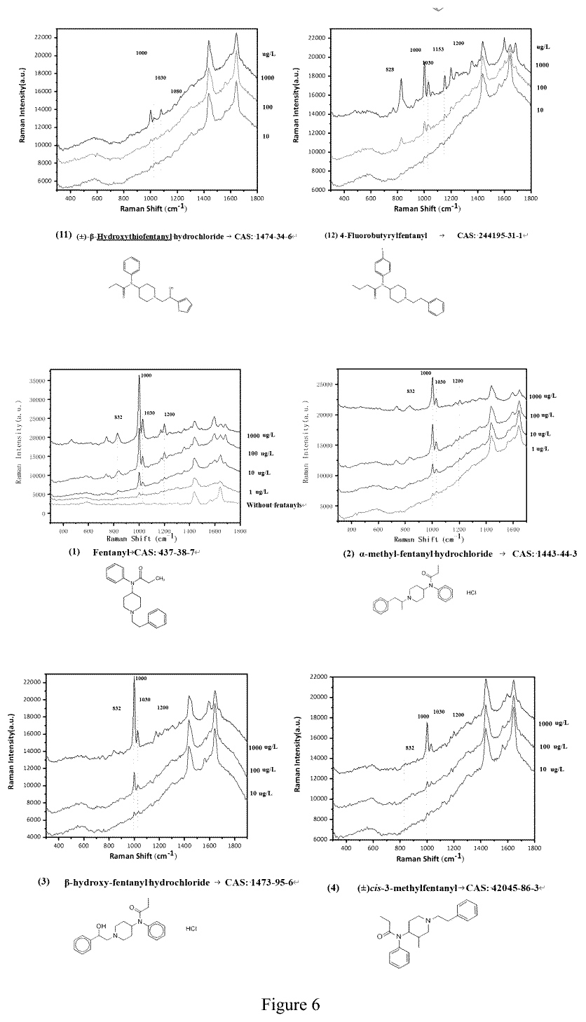

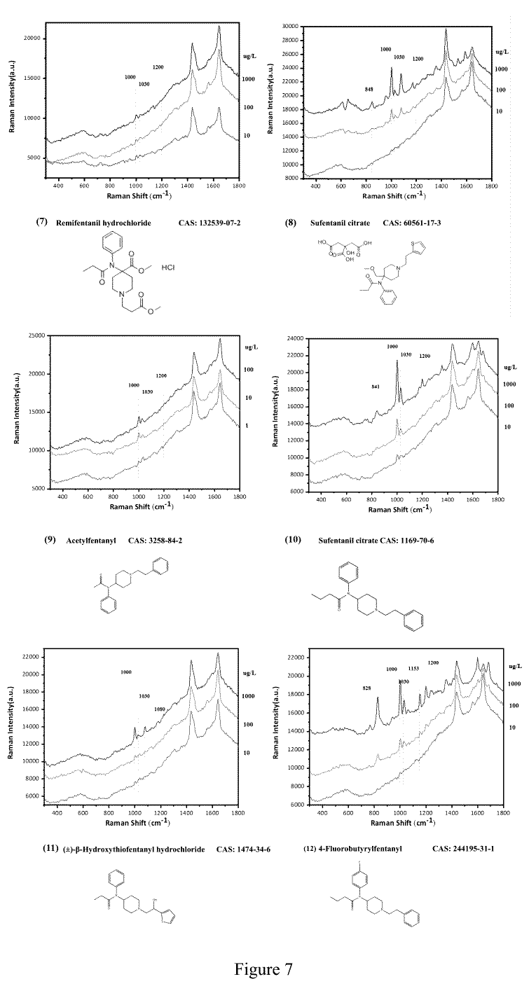

Solutions of different types of fentanyls (fentanyl, α-methyl-fentanyl hydrochloride, β-hydroxy-fentanyl hydrochloride, (±)cis-3-methylfentanyl, 3-methylthiofentanyl, p-fluorofentanyl, remifentanil hydrochloride, sufentanil citrate, acetylfentanyl, butyrylfentanyl, (±)-β-hydroxythiofentanyl hydrochloride, and 4-fluorobutyrylfentanyl) in ethanol at different concentrations (1 μg/L, 10 μg/L, 100 μg/L, or 1000 μg/L) and a blank solvent (ethanol) without fentanyls were separately added to the surface-enhanced Raman nanoparticle sol of the gold nanocake prepared according to the procedure of Example 1. The volume of the surface-enhanced Raman nanoparticle sol of the gold nanocake was 200 μL, and the addition amount of the fentanyl ethanol solution or the blank solvent (ethanol) without fentanyls was 100 μL; then 50 μL of a 1 mol/L aqueous KI solution was added. The mixture was mixed, and detected by using a Raman spectrometer; the detection conditions of the Raman spectrometer were the same as Example 2. The results are shown in FIGS. 6 and 7.

Analysis of results: From the results of FIGS. 6 and 7, it is apparent that the spectrogram of the blank solvent without fentanyls has no peak at 832±6 cm-1, 1000±6 cm-1, 1030±6 cm-1, and 1200±6 cm-1 shifts, and the spectrogram of the addition of 1 g/L, 10 μg/L, 100 μg/L, or 1000 μg/L of a fentanyl ethanol solution has at least 2 peaks at 832±2 cm-1, 1000±2 cm-1, 1030±2 cm-1, and 1200±2 cm-1 shifts. The surface-enhanced Raman nanoparticle (or surface-enhanced Raman nanoparticle sol) and the detection method provided by the present invention were used for detection, and it is known that when the surface-enhanced Raman nanoparticle (or surface-enhanced Raman nanoparticle sol) and the detection method provided by the present invention were used for detection, the detection limit for fentanyl and α-methyl-fentanyl hydrochloride was as low as 1 μg/L, and the detection limit for other fentanyls was as low as 10 μg/L.

Conclusion: The present invention researches the detection nanoparticle most suitable for detecting fentanyls by optimizing the morphology of the metal nanoparticle, and develops a method for quickly extracting fentanyls in samples such as mail or packages. The detection method of the present invention is different from other detection methods. Mass spectrometry requires professional instruments and technical personnel, and is time consuming. The detection by chromatography requires large-scale instruments and equipment, has certain requirements on experimental sites and environment, is expensive and needs to be operated by professional technical personnel. The X-ray imaging technology has weak detection capability on the novel fentanyl derivative drugs. The method of the present invention rapidly and non-destructively detects fentanyls in a sample such as mail or a package on the basis of surface-enhanced Raman. The method is convenient for experimental pretreatment operation, has low cost and high speed, and causes no damage. A portable Raman spectrometer may also be used, which is easy to carry. It is a simple method which is very suitable for on-site rapid detection.

The method of the present disclosure has been described by preferred embodiments. It will be apparent to those skilled in the art that the method and application described herein can be implemented and applied with modification or with appropriate modification and combination within the content, spirit, and scope of the present disclosure. Those skilled in the art can modify the process parameters appropriately in view of the disclosure herein. It is specifically noted that all such substitutions and modifications will be apparent to those skilled in the art and are intended to be included herein.

1. A surface-enhanced Raman nanoparticle, wherein the surface-enhanced Raman nanoparticle is a noble metal nanoparticle, the noble metal nanoparticle has a particle size of 20 nm to 200 nm, and the noble metal in the noble metal nanoparticle comprises gold or silver; the noble metal nanoparticle is one of a gold nanosphere, a gold nanocake, a gold nanorod, a gold core-shell structure, a silver nanosphere, and a silver cubic particle.

2. The surface-enhanced Raman nanoparticle according to claim 1, wherein the noble metal nanoparticle is a gold nanocake.

3. The surface-enhanced Raman nanoparticle according to claim 1, wherein the noble metal nanoparticle is a gold nanocake having a diameter of 40 nm to 100 nm and a thickness of 15 nm to 35 nm.

4. The surface-enhanced Raman nanoparticle according to claim 1, wherein the noble metal nanoparticle is a gold nanocake having a diameter of 80 nm±10 nm and a thickness of 30 nm±5 nm.

5. A surface-enhanced Raman nanoparticle sol, comprising the surface-enhanced Raman nanoparticle according to claim 1 and a solvent.

6. The surface-enhanced Raman nanoparticle sol according to claim 5, wherein the solvent comprises at least one of water, ethanol, and methanol.

7. The surface-enhanced Raman nanoparticle sol according to claim 5, wherein the surface-enhanced Raman nanoparticle has a structure of a gold nanocake, and the surface-enhanced Raman nanoparticle sol is prepared by a method comprising: mixing a noble metal precursor solution with a protectant solution, and then adding ethanol; after a reaction, obtaining a noble metal nanoparticle solution, centrifuging to obtain precipitate 1, washing the precipitate 1, centrifuging to obtain precipitate 2, and adding water to the precipitate 2 for mixing to obtain the surface-enhanced Raman nanoparticle sol.

8. The surface-enhanced Raman nanoparticle sol according to claim 5, wherein, the noble metal precursor solution is heated to 90° C. to 105° C. before being mixed with the protectant solution.

9. The surface-enhanced Raman nanoparticle sol according to claim 5, wherein the noble metal precursor in the noble metal precursor solution comprises tetrachloroauric acid (HAuCl4);
the protectant in the protectant solution comprises at least one selected from sodium citrate, polyvinylpyrrolidone (PVP), cetyltrimethylammonium bromide (CTAB), cetyltrimethylammonium chloride (CTAC), and cetyltrimethylammonium iodide (CTAI).

10. The surface-enhanced Raman nanoparticle sol according to claim 5, wherein the noble metal precursor solution and the protectant solution are mixed within 2 seconds; and the reaction has a reaction temperature of 90° C. to 105° C.

11. The surface-enhanced Raman nanoparticle sol according to claim 5, wherein a feeding mass ratio of the noble metal precursor in the noble metal precursor solution to the protectant in the protectant solution is (10.0:3.0) to (10.0:1.0).

12. The surface-enhanced Raman nanoparticle sol according to claim 5, wherein the noble metal nanoparticle in the surface-enhanced Raman nanoparticle sol has a concentration of 6×10¹¹ particles/mL to 6×10¹³ particles/mL.

13. The surface-enhanced Raman nanoparticle sol according to claim 5, wherein the surface-enhanced Raman nanoparticle has a structure of a gold nanocake, and the surface-enhanced Raman nanoparticle sol is prepared by a method comprising: heating the noble metal precursor solution to 90° C. to 105° C. and heating for 20 min to 35 min; mixing the noble metal precursor solution with the protectant solution; mixing the noble metal precursor solution and the protectant solution within 2 seconds, then adding ethanol (to adjust the morphology of the particles), wherein the ethanol has an adding amount of 10 μL, reacting at 90° C. to 105° C. for 20 min to 60 min, cooling to room temperature, obtaining a noble metal nanoparticle solution, centrifuging to obtain precipitate 1, washing the precipitate 1, centrifuging to obtain precipitate 2, and adding water to the precipitate 2 for mixing to obtain the surface-enhanced Raman nanoparticle sol; the noble metal precursor in the noble metal precursor solution comprises tetrachloroauric acid (HAuCl4); the noble metal precursor in the noble metal precursor solution has a concentration of 0.05 wt %; the protectant in the protectant solution has a concentration of 1.0 wt %; the solvent of the noble metal precursor solution comprises water; the solvent of the protectant solution comprises water; the washing is performed 1-5 times; the noble metal nanoparticle in the surface-enhanced Raman nanoparticle sol has a concentration of 6×10¹¹ particles/mL to 6×10¹³ particles/mL.

14. A detection method for fentanyls, comprising the following steps:
(1) extraction: taking a liquid sample to be detected or wiping a solid sample to be detected by using a wiping solvent;
(2) Raman spectrum detection: transferring the liquid sample to be detected or the wiping solvent after wiping into the surface-enhanced Raman nanoparticle sol according to any one of claims 2 to 5, and detecting by using a Raman spectrum with a laser wavelength of 785 nm, 633 nm, 532 nm, and/or 1064 nm to obtain Raman frequency shift spectrum data of the liquid sample to be detected;
(3) result judgment: wherein the liquid sample to be detected that has a peak at least at 1, 2, 3, or 4 positions at wave numbers of 832±6 cm-1, 1000±6 cm-1, 1030±6 cm-1, and 1200±6 cm-1 in the Raman frequency shift spectrum data comprises fentanyls; or the solid sample to be detected or the liquid sample to be detected that has a peak at least at 1, 2, or 3 positions at wave numbers of 1000±6 cm-1, 1030±6 cm-1, and 1200±6 cm-1 in the Raman frequency shift spectrum data comprises fentanyls; the solid sample to be detected or the liquid sample to be detected that has no peak at wave numbers of 832±6 cm-1, 1000±6 cm-1, 1030±6 cm-1, and 1200±6 cm-1 in the Raman frequency shift spectrum data does not comprise fentanyls.

15. The detection method according to claim 14, wherein the Raman spectrum detection in step (2) further comprises adding an agglomerant solution after transferring the liquid sample to be detected or the wiping solvent after wiping into the surface-enhanced Raman nanoparticle sol according to any one of claims 2 to 5 and before performing the detection by using Raman spectrum.

16. The detection method according to claim 14, wherein the agglomerant in the agglomerant solution comprises at least one of KCl, KBr, KI, MgSO4, MgCl2, NaBr, and NaCl.

17. The detection method according to claim 14, wherein the agglomerant in the agglomerant solution comprises KI.

18. The detection method according to claim 14, wherein the agglomerant in the agglomerant solution has a concentration of 0.01 mol/L to 5.00 mol/L.

19. The detection method according to claim 14, wherein the agglomerant solution adopts at least one of water, methanol, and ethanol as a solvent.

20. The detection method according to claim 14, wherein the fentanyls comprises at least one of 4-fluorobutyrylfentanyl, 4-fluoroisobutyrylfentanyl, butyrylfentanyl, isobutyrylfentanyl or a salt thereof, furanylfentanyl or a salt thereof, valerylfentanyl or a salt thereof, β-hydroxythiofentanyl, cis-3-methylfentanyl or a salt thereof, ocfentanil, p-fluorofentanyl, sufentanil citrate, acetylfentanyl, β-hydroxy-3-methylfentanyl, 4-anilino-N-phenylethylpiperidine, remifentanil or a salt thereof, α-methylfentanyl or a salt thereof, N-phenylethyl-4-piperidone, carfentanil, β-hydroxyfentanyl, 3-methylthiofentanyl or a salt thereof, alfentanil, fentanyl, acetyl-alpha-methylfentanyl, acrylfentanyl or a salt thereof, thiofentanyl or a salt thereof, alpha-methylthiofentanyl or a salt thereof, tetrahydrofuranylfentanyl, 2-thenoylfentanyl or a salt thereof, chloroacetylfentanyl or a salt thereof, benzoylfentanyl or a salt thereof, (2-fluorobenzoyl)fentanyl or a salt thereof, (3-fluorobenzoyl)fentanyl or a salt thereof, (2-chlorobenzoyl)fentanyl or a salt thereof, (4-fluorobenzoyl)fentanyl or a salt thereof, p-chlorofuranylformylfentanyl or a salt thereof, p-chloromethoxyacetylfentanyl or a salt thereof, p-chlorothienylformylfentanyl or a salt thereof, p-chlorobenzoylfentanyl or a salt thereof, p-chlorocyclopropylformylfentanyl or a salt thereof, p-chloroacetylfentanyl or a salt thereof, cyclopentylformylfentanyl or a salt thereof, cyclobutylformylfentanyl or a salt thereof, heptanoylfentanyl or a salt thereof, ethoxyacetylfentanyl or a salt thereof, phenylpropanoylfentanyl or a salt thereof, butyryl-alpha-methylfentanyl or a salt thereof, cyclobutylformylfentanyl or a salt thereof, isovalerylfentanyl or a salt thereof, N-benzylbutyrylfentanyl or a salt thereof, N-benzylcyclopropylfentanyl or a salt thereof, N-benzylpentanoylfentanyl or a salt thereof, N-benzylacetylfentanyl or a salt thereof, N-benzylhexanoylfentanyl or a salt thereof, alpha-methylfentanyl or a salt thereof, beta-hydroxy-3-methylfentanyl or a salt thereof, beta-hydroxyfentanyl or a salt thereof, beta-hydroxyisobutyrylfentanyl or a salt thereof, beta-hydroxyvalerylfentanyl or a salt thereof, thiofentanyl or a salt thereof, ethoxyacetylfentanyl or a salt thereof, cyclopentylformylfentanyl or a salt thereof, p-methylbutyrylfentanyl or a salt thereof, o-methylfentanyl or a salt thereof, p-methoxyacryloylfentanyl or a salt thereof, N-benzylfentanyl or a salt thereof, N-benzyl-p-fluorofentanyl or a salt thereof, norcarfentanil or a salt thereof, N-(4-methylphenylethyl)-isobutyrylfentanyl or a salt thereof, phenylpropanoylfentanyl or a salt thereof, heptanoylfentanyl or a salt thereof, 2-thienylformylfentanyl or a salt thereof, chloroacetylfentanyl or a salt thereof, benzoylfentanyl or a salt thereof, cyclobutylformylfentanyl or a salt thereof, (2-fluorobenzoyl)fentanyl or a salt thereof, (3-fluorobenzoyl)fentanyl or a salt thereof, isovaleroylfentanyl or a salt thereof, (2-chlorobenzoyl)fentanyl or a salt thereof, (4-fluorobenzoyl)fentanyl or a salt thereof, p-fluoroacetylfentanyl or a salt thereof, p-fluorotetrahydrofuranylformylfentanyl or a salt thereof, p-fluorobenzoylfentanyl or a salt thereof, p-fluorothienylformylfentanyl or a salt thereof, p-fluorofuranylformylfentanyl or a salt thereof, p-fluorocyclopentanoylformylfentanyl or a salt thereof, p-fluoropentanoylfentanyl or a salt thereof, o-fluorofentanyl or a salt thereof, o-fluoroacroloylfentanyl or a salt thereof, o-fluoroacetylfentanyl or a salt thereof, m-fluorofentanyl or a salt thereof, m-fluoromethoxyacetylfentanyl or a salt thereof, m-fluoroisobutyrylfentanyl or a salt thereof, m-fluoroacetylfentanyl or a salt thereof, m-fluorofuranylfentanyl or a salt thereof, m-fluorobenzoylfentanyl or a salt thereof, p-chlorofentanyl or a salt thereof, p-chlorobutyrylfentanyl or a salt thereof, p-chlorofuranylformylfentanyl or a salt thereof, p-chloromethoxyacetylfentanyl or a salt thereof, p-chlorothienylformylfentanyl or a salt thereof, p-chlorobenzoylfentanyl or a salt thereof, p-chlorocyclopropylformylfentanyl or a salt thereof, p-chloroacetylfentanyl or a salt thereof, p-methylfentanyl or a salt thereof, p-methyl-(4-fluorobenzoyl)fentanyl or a salt thereof, p-methylcyclopentylformylfentanyl or a salt thereof, p-methylcyclohexylformylfentanyl or a salt thereof, p-methyl-tert-butylformylfentanyl or a salt thereof, p-methylcyclopropylformylfentanyl or a salt thereof, p-methylmethoxyacetylfentanyl or a salt thereof, p-methylthienylformylfentanyl or a salt thereof, p-methylfuranylformylfentanyl or a salt thereof, p-methylbenzoylfentanyl or a salt thereof, p-methylethoxyacetylfentanyl or a salt thereof, p-methyltetrahydrofuranylformylfentanyl or a salt thereof, p-methyl-(4-chlorobenzoyl)fentanyl or a salt thereof, o-methylfuranylformylfentanyl or a salt thereof, o-methylcyclohexylformylfentanyl or a salt thereof, o-methylbutyrylfentanyl or a salt thereof, o-methylbenzoylfentanyl or a salt thereof, o-methyl-(4-fluorobenzoyl)fentanyl or a salt thereof, o-methylthienylformylfentanyl or a salt thereof, o-methylcyclopentylformylfentanyl or a salt thereof, benzoylalpha-methylfentanyl or a salt thereof, hexanoylalpha-methylfentanyl or a salt thereof, p-methoxytetrahydrofuranylfentanyl or a salt thereof, p-methoxyisobutyrylfentanyl or a salt thereof, p-methoxy-2-methoxyacetylfentanyl or a salt thereof, p-methoxyhexanoylfentanyl or a salt thereof, thioacetylfentanyl or a salt thereof, N-benzylacetylfentanyl or a salt thereof, N-benzylbutyrylfentanyl or a salt thereof, N-benzyl-(4-chlorobenzoyl)fentanyl or a salt thereof, N-benzylcyclopropylfentanyl or a salt thereof, N-benzylcyclopentylfentanyl or a salt thereof, N-benzylvalerylfentanyl or a salt thereof, N-benzylfuranylfentanyl or a salt thereof, N-benzylhexanoylfentanyl or a salt thereof, N-benzyl-p-fluorocyclopentylformylfentanyl or a salt thereof, N-benzyl-p-fluoro-cyclohexylformylfentanyl or a salt thereof, N-benzyl-p-fluoro-furanylformylfentanyl or a salt thereof, N-benzyl-p-fluoro-(3-fluorobenzoyl)fentanyl or a salt thereof, N-benzyl-p-fluoro-acetylfentanyl or a salt thereof, N-benzyl-p-fluoro-thienylformylfentanyl or a salt thereof, N-benzyl-p-fluoro-methoxyacetylfentanyl or a salt thereof, N-benzyl-p-fluoro-isobutyrylfentanyl or a salt thereof, N-benzyl-p-fluoro-butyrylfentanyl or a salt thereof, N-benzyl-p-fluoro-(4-chlorobenzoyl)fentanyl or a salt thereof, N-methylbenzoylfentanyl or a salt thereof, N-methyl-(4-chlorobenzoyl)fentanyl or a salt thereof, N-methyl-(2-fluorobenzoyl)fentanyl or a salt thereof, N-(4-methylphenylethyl)-benzoylfentanyl or a salt thereof, N-(4-methylphenylethyl)fentanyl or a salt thereof, N-(4-methylphenylethyl)-isovalerylfentanyl or a salt thereof, N-(4-methylphenylethyl)-n-butyrylfentanyl or a salt thereof, N-cyclopropylformylcyclopropylfentanyl or a salt thereof, p-fluorofentanyl or a salt thereof, N-(4-methylphenylethyl)-(4-chlorobenzoyl)fentanyl or a salt thereof, N-(4-methylphenylethyl)-thienylformylfentanyl or a salt thereof, N-(4-nitrophenylethyl)fentanyl or a salt thereof, valerylalpha-methylfentanyl or a salt thereof, N-benzylisobutyrylfentanyl or a salt thereof, N-(4-methylphenylethyl)-cyclohexylformylfentanyl or a salt thereof, N-(4-chlorophenylethyl)fentanyl or a salt thereof, N-(4-methylphenylethyl)-methoxyacetylfentanyl or a salt thereof, N-(4-methylphenylethyl)-acetylfentanyl or a salt thereof, o-methoxybutyrylfentanyl or a salt thereof, o-methoxyvalerylfentanyl or a salt thereof, 3-methylthioacetylfentanyl or a salt thereof, butyrylalpha-methylfentanyl or a salt thereof, betahydroxy-3-methylbutyrylfentanyl or a salt thereof, and 3-methylthiobutyrylfentanyl or a salt thereof.
